(19) United States
(12) Patent Application Publication
(10) Pub. No.: US 2014/0079604 A1
(43) Pub. Date: Mar. 20, 2014

(54) APPARATUS FOR FACILITATING EXAMINATION OF TISSUE SAMPLES

(71) Applicant: Patrick O'Neill, Apex, NC (US)

(72) Inventor: Patrick O'Neill, Apex, NC (US)

(21) Appl. No.: 13/621,280

(22) Filed: Sep. 16, 2012

(51) Int. Cl. G01N 33/48

(52) U.S. Cl. 422/536

(57) ABSTRACT

Apparatuses for facilitating examination of tissue samples are provided. In accordance with an exemplary embodiment, a cassette for preparing a tissue specimen for examination includes a base having a continuous curvilinear wall that forms a cavity. The cavity is configured for receiving the tissue specimen and defines an opening. Further, the base forms a plurality of apertures in the curvilinear wall that are configured to allow fluid flow into and out of the cavity. Also, the cassette includes a cover that is configured to enclose the cavity.

### TECHNICAL FIELD

The present invention generally relates to the histological examination of tissue samples, and more particularly relates to a cassette for holding a tissue sample during preparation for histological examination.

### BACKGROUND

Tissue block preparation is a technique used to prepare embedded biological samples for analysis. In the tissue block procedure, tissue samples are immobilized in a solid or semi-solid support, such as paraffin or wax. Often, the tissue samples are inserted in a recipient paraffin block in a precisely spaced, array pattern. Sections from the paraffin block can then be cut using a microtome, mounted on a microscope slide, and analyzed by any method of standard histological analysis. Each microarray block can be cut into one hundred to five hundred sections, which can be subjected to independent tests. Tests commonly employed in tissue microarray include immunohistochemistry and fluorescent in situ hybridization. Tissue microarrays are particularly useful in analysis of cancer samples.

As noted, the tissue block preparation method requires that the tissue samples be “embedded” in a solid or semi-solid medium, most commonly paraffin wax. “Embedding” typically requires the following steps: (1) all water molecules must be removed from the tissue samples, typically by alcohol (water is miscible with alcohol); (2) all alcohol must then be removed, as well as all fatty substances, and replaced typically by xylene (xylene is miscible with alcohol but not water); (3) the xylene must be removed and replaced with wax (wax is miscible with xylene but not with most alcohols or water); and (4) the tissue samples in molten wax must then be manually organized and hardened so that a section of the wax block with the embedded tissue can be cut using a microtome.

It is desirable to use less invasive techniques which take smaller tissue samples for histological examination. However, the use of smaller tissue samples presents mechanical difficulties in the preparation method. Specifically, smaller tissue samples are more difficult to locate and to move from the preparation location for embedding in the semi-solid medium.

Accordingly, it is desirable to provide apparatuses for facilitating examination of tissue samples. In addition, it is desirable to provide apparatuses for holding tissue samples during pretreatment before use in a tissue block. Furthermore, other desirable features and characteristics of the present invention will become apparent from the subsequent detailed description of the invention and the appended claims, taken in conjunction with the accompanying drawings and this background of the invention.

### BRIEF SUMMARY

Apparatuses for preparing tissue specimens for examination are provided. In accordance with an exemplary embodiment, a cassette for preparing a tissue specimen for examination includes a base having a continuous curvilinear wall. The continuous curvilinear wall forms a cavity configured for receiving the tissue specimen. Further, the wall defines an opening. The base also forms a plurality of apertures in the curvilinear wall that are configured to allow fluid flow into and out of the cavity. The cassette is provided with a cover that is configured to enclose the cavity.

In accordance with another exemplary embodiment, a cassette for holding a tissue specimen during pretreatment before examination includes a continuous curvilinear wall that defines a concave cavity configured for receiving the tissue specimen. The curvilinear wall terminates at an opening and includes a plurality of apertures that are configured to allow fluid flow into and out of the cavity. Further, the cassette is provided with a support structure including a substantially planar top wall that extends outwardly from the opening. The support structure includes a bottom that defines a bottom plane substantially parallel to the top wall. The cassette also includes a cover that is configured to enclose the cavity.

In accordance with another exemplary embodiment, a cassette is provided for preparing a tissue specimen for examination. The cassette comprises a unitary body that forms a continuous curvilinear wall integrated into a support structure. The continuous curvilinear wall has a bottom surface and a top surface that defines a cavity configured for receiving the tissue specimen. Further, the continuous curvilinear wall defines an opening in communication with the cavity and is formed with a plurality of apertures configured to allow fluid flow into and out of the cavity. The support structure defines at least one channel to allow fluid flow to the bottom surface of the continuous curvilinear wall.

### BRIEF DESCRIPTION OF THE DRAWINGS

The present invention will hereinafter be described in conjunction with the following drawing figure wherein:

FIG. 1 is a perspective view of the cassette in an open configuration, showing the top of the base and the underside of the cover in accordance with an exemplary embodiment;

FIG. 2 is a perspective view of the cassette of FIG. 1 in the open configuration, showing bottom of the base and the top side of the cover in accordance with an exemplary embodiment.

FIG. 3 is a top view of the cassette of FIG. 1 in a closed configuration, showing the top side of the cover in accordance with an exemplary embodiment;

FIG. 4 is a side cross sectional view of the cassette of FIG. 1 in the open configuration in accordance with an exemplary embodiment,

FIG. 5 is a top perspective view of an alternate embodiment of the base of FIG. 1;

FIG. 6 is a bottom perspective view of the base of FIG. 5; and

FIG. 7 is a top perspective view of an alternate embodiment of the cassette in a closed configuration.

### DETAILED DESCRIPTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to a cassette for holding a tissue sample during pretreatment before examination. Pretreatment generally involves contact of the tissue sample with various liquids, and may include flowing liquids through the cassette or immersing the cassette in liquids. As a result, the tissue sample is held within an enclosed cavity that is formed with apertures. The apertures allow for fluid flow into and out of the cavity. After treatment, the tissue sample may be removed from the cavity. Importantly, the cavity is provided with a continuous curvilinear wall to enable easy removal of the tissue sample.

In FIG. 1, a cassette for preparing a tissue specimen for examination is shown and generally designated 10. As shown, the cassette 10 includes a unitary base 12 that is formed with a continuous curvilinear wall 14 integrated into a support structure 16. As used herein, unitary means that the base is of one-piece construction, such as a molded or forged piece, that includes no joints or connections between its components, the wall 14 and the support structure 16. Further, as used herein continuous curvilinear means that the wall 14 includes a top surface 15 having no corners or junctions. Instead, the continuous curvilinear wall 14 terminates at a substantially oval-shaped opening 18. As shown, the wall 14 defines a concave cavity 20 that defines a volume in the shape of a portion of a spheroid. Along its long axis, the cavity 20 has a length to depth ratio of between about 5:1 to 10:1, and preferably about 7.5:1. The cavity 20 terminates at a plane 22 defined by the opening 18. In FIG. 1, the wall 14 is shown formed with a plurality of apertures 24 for allowing fluid to flow into and out of the cavity 20. As shown, each aperture 24 defines an aperture axis, and the aperture axes converge at a location above the opening 18.

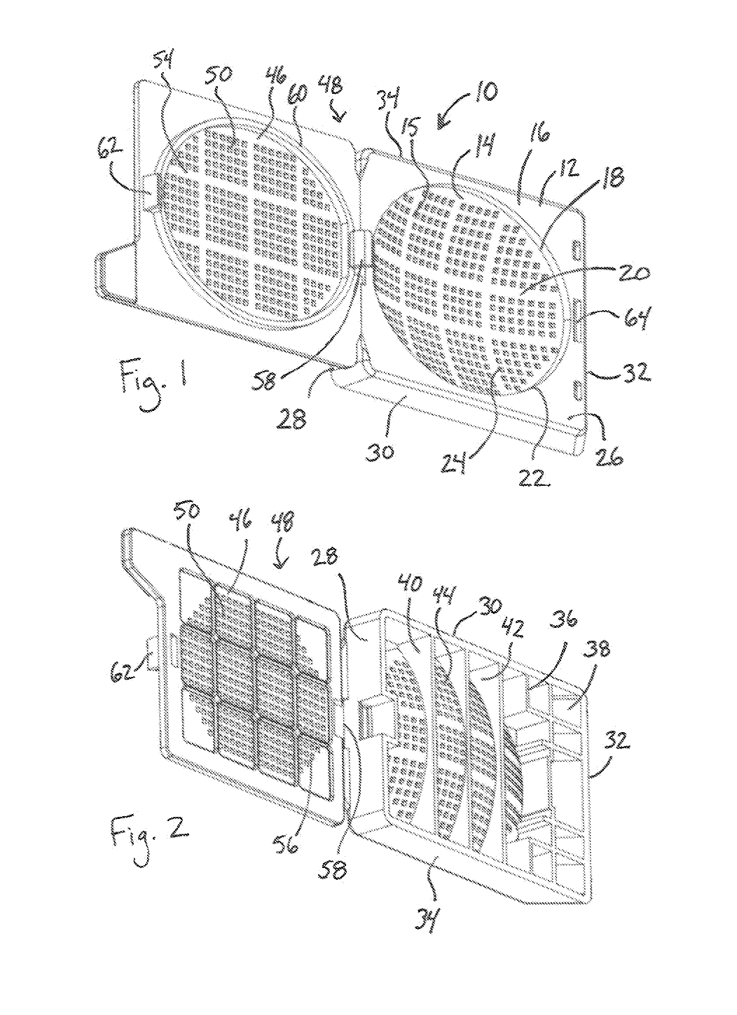

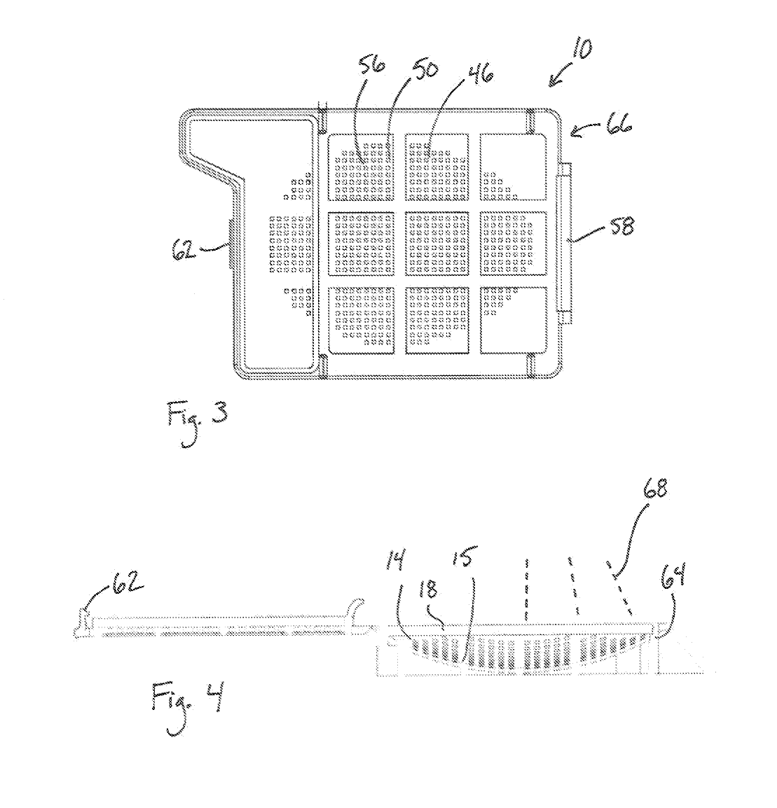

The support structure 16 includes a substantially planar top wall 26 that extends outwardly from the opening 18 along the plane 22 to side walls 28, 30, 32 and 34. In addition to the top wall 26 and side walls 28, 30, 32 and 34, the support structure 16 includes a bottom 36 as shown in FIG. 2. The bottom 36 defines a bottom plane 38 that is substantially parallel to the plane 22. As shown, the bottom 36 is formed by the termination of the side walls 28, 30, 32, and 34, as well as by the ends of supports 40. In FIG. 2, it can be seen that the continuous curvilinear wall 14 is integrated into the supports 40. Further, it can be seen that the support structure 16 forms channels 42 that allow fluid flow to the bottom surface 44 of the curvilinear wall 14.

Cross-referencing FIGS. 1 and 2, it can be seen that the cassette 10 includes a substantially planar cover 46. In both FIGS. 1 and 2, the cassette is in an open configuration 48 such that the cavity 20 is not covered. This configuration 48 is useful for introducing and removing tissue samples from the cavity 20. As shown, the cover 46 is formed with a plurality of apertures 50 configured to allow fluid flow into and out of the cavity 20 when in a closed configuration. The cover 46 has a bottom side 54 and a top side 56. Further, the cover 46 is pivotably attached to the base 12 by a joint 58. As shown, the cover 46 includes a rim wall 60 that may be received within the cavity 20 when the cassette 10 is in a closed configuration. Also, the cover 46 and base 12 are provided with engagement members 62 and 64 as a means to secure the cover 46 when in its closed configuration. The engagement members 62 and 64 provide for a releasable mechanical engagement.

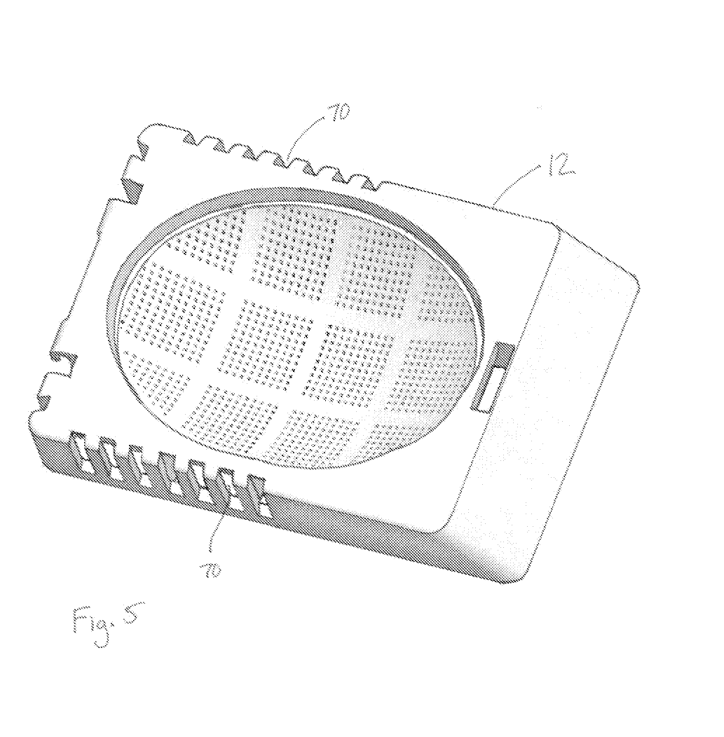

Referring now to FIG. 3, the cassette 10 is shown its closed configuration 66. During use, the tissue sample is placed within the cavity 20 and the cover 46 is moved to the closed configuration 66 to enclose the cavity 20. The engagement members 62 and 64 secure the cassette 10 in the closed configuration 66 while the tissue sample is treated with a liquid or liquids. After treatment, the engagement members 62 and 64 are disengaged and the cover 46 is moved to the open configuration 48. As shown in FIG. 4, the top surface 15 of the curvilinear wall 14 includes no corners or junctions where a tissue sample can be difficult to trap and collect. As further shown in FIG. 4, the apertures 24 define aperture axes 68 that converge above the opening 18.

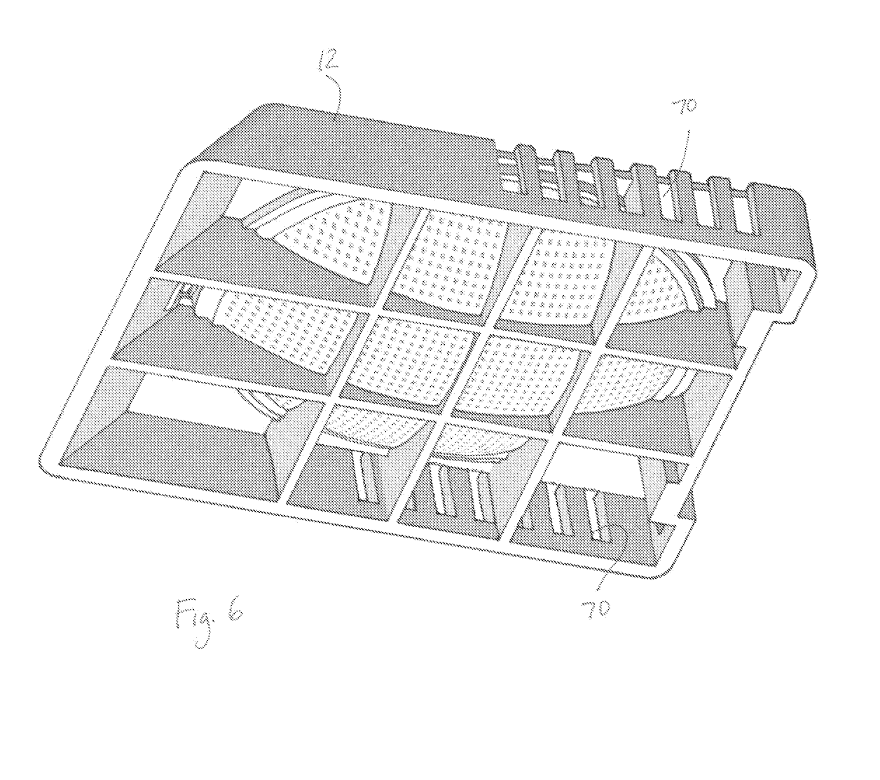

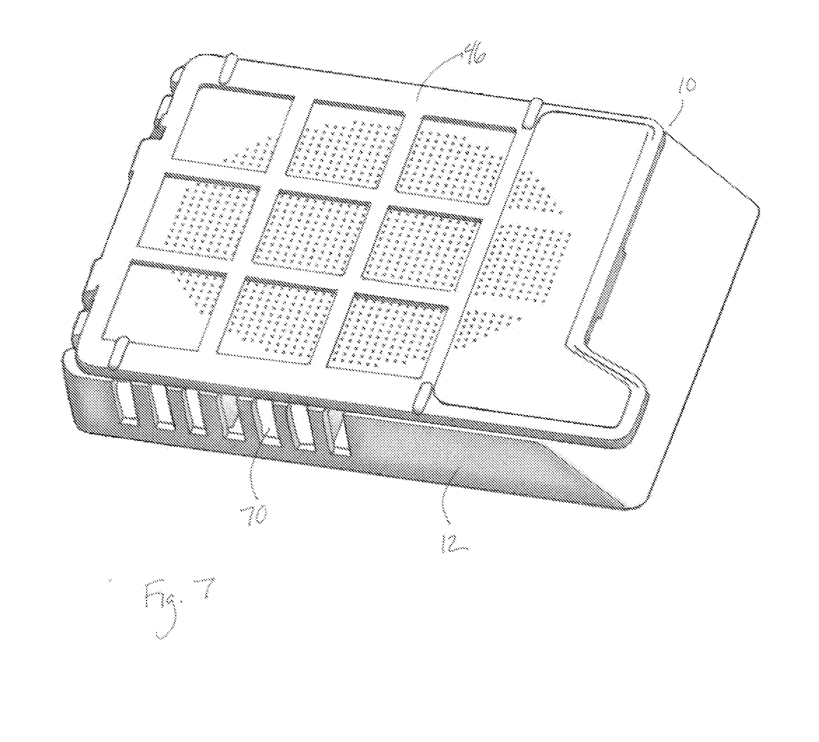

In FIGS. 5-7, an alternate embodiment of the base 12 is shown. The base 12 includes vents or holes 70 to allow for the passage of fluid to and from the bottom surface 44 of the curvilinear wall 14.

Accordingly, various embodiments relating to a cassette for preparing a tissue sample for examination have been provided. While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cassette for preparing a tissue specimen for examination comprising:
a base having a continuous curvilinear wall forming a cavity configured for receiving the tissue specimen and defining an opening, wherein the base forms a plurality of apertures in the curvilinear wall configured to allow fluid flow into and out of the cavity; and
a cover configured to enclose the cavity.

2. The cassette of claim 1 wherein the opening defines a plane and wherein the opening is oval shaped.

3. The cassette of claim 1 wherein the cavity defines a volume in the shape of a portion of a spheroid.

4. The cassette of claim 1 wherein the cover forms a plurality of apertures configured to allow fluid flow into and out of the cavity.

5. The cassette of claim 1 wherein the cover is pivotably connected to the base.

6. The cassette of claim 1 further comprising a substantially planar top wall extending outwardly from the opening, wherein the top wall includes a means for pivotable attachment to the cover, and wherein the top wall includes a means for selectively securing the cover to the top wall in a closed configuration.

7. The cassette of claim 1 wherein the cover defines a rim wall that is received within the cavity when the cover is in a closed configuration.

8. The cassette of claim 1 wherein the cavity has a length to depth ratio of about 7.5:1.

9. The cassette of claim 1 wherein each of the apertures defines an aperture axis, and wherein the aperture axes converge above the opening.

10. A cassette for holding a tissue specimen during pretreatment before examination comprising:
a continuous curvilinear wall defining a concave cavity configured for receiving the tissue specimen, wherein the curvilinear wall terminates at an opening, and wherein a plurality of apertures are formed in the curvilinear wall and are configured to allow fluid flow into and out of the cavity;
a support structure including a substantially planar top wall extending outwardly from the opening, and a bottom defining a bottom plane substantially parallel to the top wall; and
a cover configured to enclose the cavity.

11. The cassette of claim 10 wherein the top wall includes a means for pivotable attachment to the cover, and wherein the top wall includes a means for selectively securing the cover to the top wall in a closed configuration.

12. The cassette of claim 10 wherein the opening defines a plane and wherein the opening is oval shaped.

13. The cassette of claim 10 wherein the cavity defines a volume in the shape of a portion of a spheroid.

14. The cassette of claim 10 wherein the cover forms a plurality of apertures configured to allow fluid flow into and out of the cavity.

15. The cassette of claim 10 wherein the cover is pivotably connected to the base.

16. The cassette of claim 10 wherein the cover defines a rim wall that is received within the cavity when the cover is in a closed configuration.

17. The cassette of claim 1 wherein the cavity has a length to depth ratio of about 7.5:1.

18. The cassette of claim 1 wherein each of the apertures defines an aperture axis, and wherein the aperture axes converge above the opening.

19. A cassette for preparing a tissue specimen for examination comprising a unitary body forming a continuous curvilinear wall integrated into a support structure, wherein the continuous curvilinear wall has a bottom surface and a top surface defining a cavity configured for receiving the tissue specimen, wherein the continuous curvilinear wall defines an opening in communication with the cavity, wherein the curvilinear wall forms a plurality of apertures configured to allow fluid flow into and out of the cavity, and wherein the support structure defines at least one channel to allow fluid flow to the bottom surface of the continuous curvilinear wall.

20. The cassette of claim 19 further comprising a cover mounted to the support structure and movable between a closed configuration in which the cover encloses the cavity and an open configuration.
